(19) United States
(12) Patent Application Publication
(10) Pub. No.: US 2022/0323219 A1
(43) Pub. Date: Oct. 13, 2022

(54) ANCHORS FOR MITRAL CHORDAE REPAIR

(71) Applicant: W. L. Gore & Associates, Inc., Newark, DE (US)

(72) Inventors: Jonathan D. Combe, Phoenix, AZ (US); Thomas R. Geary, Jr., Phoenix, AZ (US); Jake M. Golden, Phoenix, AZ (US); Patrick M. Norris, Flagstaff, AZ (US); Anette I. Pico, Phoenix, AZ (US); Susan J. Rudes, Phoenix, AZ (US); Martin J. Sector, Phoenix, AZ (US); Matthew J. Zukaitis, Phoenix, AZ (US)

(21) Appl. No.: 17/762,839

(22) PCT Filed: Sep. 23, 2020
(86) PCT No.: PCT/US2020/052163

§ 371 Date: Mar. 23, 2022

Related U.S. Application Data
(60) Provisional application No. 62/904,327, filed on Sep. 23, 2019.

(51) Int. Cl. A61F 2/24

(52) U.S. Cl. CPC A61F 2/2457; A61F 2220/0016; A61F 2220/0075; A61F 2230/0069; A61F 2230/0091; A61F 2230/0093

(57) ABSTRACT

Various aspects of the present disclosure are directed toward apparatuses, systems, and methods that include tissue anchors such as for chordae tendineae repair.

### CROSS-REFERENCE TO RELATED APPLICATION

This application is a national phase application of PCT Application No. PCT/US2020/052163, internationally filed on Sep. 23, 2020, which claims the benefit of Provisional Application No. 62/904,327, filed Sep. 23, 2019, which are incorporated herein by reference in their entireties for all purposes.

### FIELD

The present disclosure relates generally to apparatuses, systems, and methods that include anchors for medical devices. More specifically, the disclosure relates to apparatuses, systems, and methods that include anchors for chordae tendineae repair.

### BACKGROUND

Leaflets of atrioventricular valves (mitral and tricuspid) are thin, diaphanous structures that rely on a system of long, thin, cord-like supports to maintain competence of the valve in the loaded condition. These supports, chordae tendineae, attach the papillary muscles to the valve leaflets.

Chordae tendineae can degenerate and stretch, which can result in leaflet prolapse. As a result, the leaflet(s) can misalign under systolic loading. An open surgical procedure for chordae tendineae is highly invasive and carries with it a high morbidity and mortality risk. Thus, delivery and implantation of artificial chordae tendineae(s) in chordae tendineae replacement or repair without using an open surgical procedure (or a transapical or transatrial delivery approach) can reduce morbidity and mortality risk.

### SUMMARY

According to one example (“Example 1”), a tubular member having a proximal end and a distal end; at least one wire arranged through the tubular member to form: a plurality of anchors extending from the distal end of the tubular member, each anchor defining a curve extending in a direction toward the proximal end, and an eyelet extending from the proximal end; and a tissue piercing element extending from the proximal end of the tubular member and arranged adjacent to the plurality of anchors.

According to another example (“Example 2”), further to the tissue anchor of Example 1, the at least one wire includes at least two wires arranged through the tubular member, and the at least two wires overlap to form the eyelet.

According to another example (“Example 3”), further to the tissue anchor of Example 2, the at least two wires form the plurality of anchors and the plurality of anchors includes at least four anchors.

According to another example (“Example 4”), further to the tissue anchor of any one of Examples 1-2, the tubular member includes a plurality of lumens extending between the proximal end and the distal end, and the lumens are configured to maintain an arrangement of the at least one wire.

According to another example (“Example 5”), further to the tissue anchor of any one of Examples 1-4, the tissue piercing element includes a longitudinal portion and a depth indicator configured to provide tactile feedback to a physician embedding of the plurality of anchors within tissue.

According to another example (“Example 6”), further to the tissue anchor of Example 5, the depth indicator includes a spacer and a biasing member arranged about the longitudinal portion of the tissue piercing element, the spacer is configured to contact tissue and urge the biasing member toward the distal end of the tubular member, the degree of which is configured to indicate the extent to which the plurality of anchors extend within the tissue.

According to another example (“Example 7”), further to the tissue anchor of any one of Examples 1-6, the tubular member comprises at least one of PEEK and stainless steel.

According to another example (“Example 8”), further to the tissue anchor of any one of Examples 1-7, the tissue anchor also includes a flexible cord arranged through the eyelet for coupling the tissue anchor to tissue.

According to another example (“Example 9”), further to the tissue anchor of any one of Examples 1-8, the tissue anchor also includes a location feedback mechanism arranged at the distal end of the tubular member and configured to indicate a slope or angle of a tissue wall.

According to another example (“Example 10”), a tissue anchor includes at least one wire arranged to form: an upper circular portion configured to interface with a flexible cord, one or more tissue piercing elements configured to engage a tissue surface and move between a first configuration in which the one or more tissue piercing elements are arranged substantially parallel to the tissue surface and a second configuration where the one or more tissue piercing elements are arranged non-parallel to the tissue surface, and an intermediate portion configured to move the one or more tissue piercing elements from the first configuration to the second configuration in response to a reduction in diameter of the middle portion.

According to another example (“Example 11”), further to the tissue anchor of Example 10, the middle portion is substantially oval in shape.

According to another example (“Example 12”), further to the tissue anchor of any one of Examples 10-11, the at least one wire includes a second circular portion and the one or more tissue piercing elements extend from the second circular portion.

According to another example (“Example 13”), further to the tissue anchor of any one of Examples 10-12, the tissue anchor also includes a delivery sheath configured to reduce the intermediate portion in diameter and unconstrain the intermediate portion allow movement of the one or more tissue piercing elements from the first configuration to the second configuration.

According to another example (“Example 14”), further to the tissue anchor of Example 13, the one or more tissue piercing elements are configured to grip the tissue surface in response to movement from the first configuration to the second configuration.

According to another example (“Example 15”), further to the tissue anchor of any one of Examples 10-14, the flexible cord is configured for coupling the tissue anchor to tissue.

According to one example (“Example 16”), a tissue anchor includes a cylindrical portion having a longitudinal axis, a proximal end and a distal end; and a plurality of anchors configured to secure the cylindrical portion under a tissue surface, each of the plurality of anchors including: a substantially linear section extending from the distal end of the cylindrical portion parallel to the longitudinal axis, and a curved section extending from the substantially linear section and configured to align with the substantially linear section relative to the longitudinal axis in a delivery configuration and curve radially outwardly relative to the longitudinal axis and toward the distal end of the cylindrical portion in a deployed configuration.

According to another example (“Example 17”), further to the tissue anchor of Example 16, the cylindrical portion includes one or more notches in an exterior surface of the cylindrical portion.

According to another example (“Example 18”), further to the tissue anchor of Example 17, the one or more notches are configured to facilitate flexibility of the cylindrical portion.

According to another example (“Example 19”), further to the tissue anchor of any one of Examples 16-18, the tissue anchor also includes a flexible cord arranged through an opening in the cylindrical portion configured for coupling the tissue anchor to tissue.

According to another example (“Example 20”), further to the tissue anchor of Example 19, the cylindrical portion includes an adjustment mechanism configured to adjust a length of the flexible cord.

According to one example (“Example 21”), a tissue anchor includes a first anchor section including a first curved section and a first tissue piercing element extending longitudinally from an interior surface of an apex the first curved section; a second anchor section including a second curved section and a second tissue piercing element extending longitudinally from an interior surface of an apex of the second curved section; a first transition section arranged between and extending perpendicular to the first anchor section and the second anchor section in a first plane; and a second transition section arranged between and extending perpendicular to the first anchor section and the second anchor section in a second plane.

According to another example (“Example 22”), further to the tissue anchor of Example 21, the first plane and the second plane are spaced apart from one another.

According to another example (“Example 23”), further to the tissue anchor of any one of Examples 21-22, at least one of the first transition section and the second transition defines a curve.

According to another example (“Example 24”), further to the tissue anchor of any one of Examples 21-23, the first anchor section, the second anchor section, the first transition section, and the second transition are configured to deploy to within a substantially common plane.

According to another example (“Example 25”), further to the tissue anchor of any one of Examples 21-24, the tissue anchor also includes a flexible cord coupled to at least one of the first anchor section, the second anchor section, the first transition section, and the second transition for coupling the tissue anchor to tissue.

According to one example (“Example 26”), a tissue anchor includes a cylindrical portion having a lumen, a proximal end and a distal end; a plurality of anchors extending from the lumen and radially outwardly relative to an exterior surface of the cylindrical portion; a ball arranged at or adjacent to the distal end of the cylindrical portion; and a flexible cord extending within the lumen of the cylindrical portion and coupled to the ball, the flexible cord being configured to withdraw the ball toward the proximal end and engage the plurality of anchors to alter a configuration of the plurality of anchors.

According to another example (“Example 27”), further to the tissue anchor of Example 26, the ball is configured to at least partially withdraw into the lumen to alter the configuration of the plurality of anchors.

According to one example (“Example 28”), a tissue anchor includes a cylindrical portion having a proximal end and a distal end; and an anchor element configured to secure the cylindrical portion to or under a tissue surface, the anchor element having a helical structure having a proximal end arranged at the distal end of the cylindrical portion and including a plurality of loops of increasing diameter from the proximal end of the helical structure to a distal end of the helical structure.

According to another example (“Example 29”), further to the tissue anchor of Example 28, the tissue anchor also includes one or more barbs arranged on an exterior surface of the helical structure.

According to another example (“Example 30”), further to the tissue anchor of any one of Examples 28-29, the tissue anchor also includes a flexible cord coupled to the cylindrical portion configured for coupling the tissue anchor to tissue.

According to one example (“Example 31”), a tissue anchor includes a cylindrical portion having a proximal end and a distal end; a plurality of substantially linear sections extending from the distal end of the cylindrical portion; and a plurality of helical anchors arranged at distal ends of each of the plurality of substantially linear sections configured to secure the cylindrical portion to a tissue wall.

According to another example (“Example 32”), further to the tissue anchor of Example 31, the tissue anchor also includes a flexible cord coupled to the cylindrical portion configured for coupling the tissue anchor to tissue.

According to one example (“Example 33”), a method for chordae tendineae repair includes: arranging a flexible cord through a leaflet and anchoring a first end of the flexible cord to the leaflet; coupling a second end of the flexible cord to the tissue anchor; and anchoring the tissue anchor of any one of Examples 1-32 to tissue of a heart.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of a patient's heart and chorda tendineae in accordance with an embodiment.

FIG. 2 is an illustration of an example chordae tendineae repair device in accordance with an embodiment.

FIG. 3A is a first perspective view of an illustration of an example tissue anchor in accordance with an embodiment.

FIG. 3B is a second perspective view of the tissue anchor, shown in FIG. 3A, in accordance with an embodiment.

FIG. 3C is a third perspective view of the tissue anchor, shown in FIGS. 3A-B, in accordance with an embodiment.

FIG. 4A is a side view of an illustration of another example tissue anchor in accordance with an embodiment.

FIG. 4B is an illustration of the tissue anchor, shown in FIG. 4A, in a delivery configuration in accordance with an embodiment.

FIG. 4C is an illustration of the tissue anchor, shown in FIGS. 4A-B, in a deployed configuration in accordance with an embodiment.

FIG. 5 is a first perspective view of an illustration of an example tissue anchor in accordance with an embodiment.

FIG. 6A is an illustration of an example tissue anchor in a deployed configuration, in accordance with an embodiment.

FIG. 6B is an illustration of the tissue anchor, shown in FIG. 6A, in a delivery configuration in accordance with an embodiment.

FIG. 6C is an illustration of the tissue anchor, shown in FIGS. 6A-B, with a flexible cord pivot in accordance with an embodiment.

FIG. 6D is an illustration of the tissue anchor, shown in FIGS. 6A-C, with an adjustment mechanism in accordance with an embodiment.

FIG. 7A is a perspective view of an illustration of another example tissue anchor in accordance with an embodiment.

FIG. 7B is a perspective view of the tissue anchor, shown in FIG. 7A, arranged with a needle in accordance with an embodiment.

FIG. 7C is a deployed view of the tissue anchor, shown in FIGS. 7A-B, in accordance with an embodiment.

FIG. 8A is an illustration of a tissue anchor in a first configuration, in a delivery configuration in accordance with an embodiment.

FIG. 8B is an illustration of the tissue anchor, shown in FIG. 8A, in a second configuration in accordance with an embodiment.

FIG. 8C is an illustration of a cut pattern for a portion of the tissue anchor, shown in FIGS. 8A-B, in accordance with an embodiment.

FIG. 9A is a perspective view of an illustration of another example tissue anchor in accordance with an embodiment.

FIG. 9B is a perspective view of a portion of the tissue anchor, shown in FIG. 9A, with one or more barbs in accordance with an embodiment.

FIG. 10 is a perspective view of an illustration of another example tissue anchor in accordance with an embodiment.

FIG. 11 is a view of an example cut-pattern for a tissue anchor in accordance with an embodiment.

FIG. 12A is a side view of an example tubular member for a tissue anchor in accordance with an embodiment.

FIG. 12B is a proximal end view of the tubular member, shown in FIG. 12A, in accordance with an embodiment.

FIG. 12C is a distal end view of the tubular member, shown in FIGS. 12A-B, in accordance with an embodiment.

FIG. 12D is a side view of another example configuration of the tubular member, shown in FIGS. 12A-C, in accordance with an embodiment.

FIG. 13A is a proximal end view of an example tubular member and at least one wire arranged through the tubular member, in accordance with an embodiment.

FIG. 13B is a proximal end view of the tubular member, shown in FIG. 13A, and another arrangement of the at least one wire arranged through the tubular member, in accordance with an embodiment.

FIG. 14A is a side view of an example tissue anchor and depth indicator, in accordance with an embodiment.

FIG. 14B is a side view of the tissue anchor, shown in FIG. 14A, and another depth indicator, in accordance with an embodiment.

FIG. 15A is a distal end view of an example tubular member and a location feedback mechanism, in accordance with an embodiment. The tissue anchor 300 may

FIG. 15B is a view of the tubular member and the location feedback mechanism, shown in FIG. 15A, engaging a tissue wall, in accordance with an embodiment.

### DETAILED DESCRIPTION

### Definitions and Terminology

This description is not meant to be read in a restrictive manner. For example, the terminology used in the description should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures. Indeed, the referenced components may be oriented in any direction. Similarly, throughout this disclosure, where a process or method is shown or described, the method may be performed in any order or simultaneously, unless it is clear from the context that the method depends on certain actions being performed first.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Applications for the methods, systems, and devices discussed herein may be directed toward providing an artificial chordae that includes a flexible cord. The artificial chordae may be configured to be attached to one or more valve leaflets and/or other heart structures, such as one or more heart wall(s) (e.g., septums or other chamber walls), papillary muscles, or other structures. In some applications, the valve undergoing repair may be the mitral valve or tricuspid valve. In various examples, the flexible cord is coupled at the superior end to one or more leaflets and at the inferior end to the papillary muscle, ventricular wall, or other structure. One or both ends of the flexible cord may include an anchor for coupling the flexible cord between heart structures. Although various embodiments are described in association with chordae tendinea applications, it should be appreciated that it is specifically contemplated that the principles of operation and associated features and concepts are applicable to any tissue anchoring application as desired.

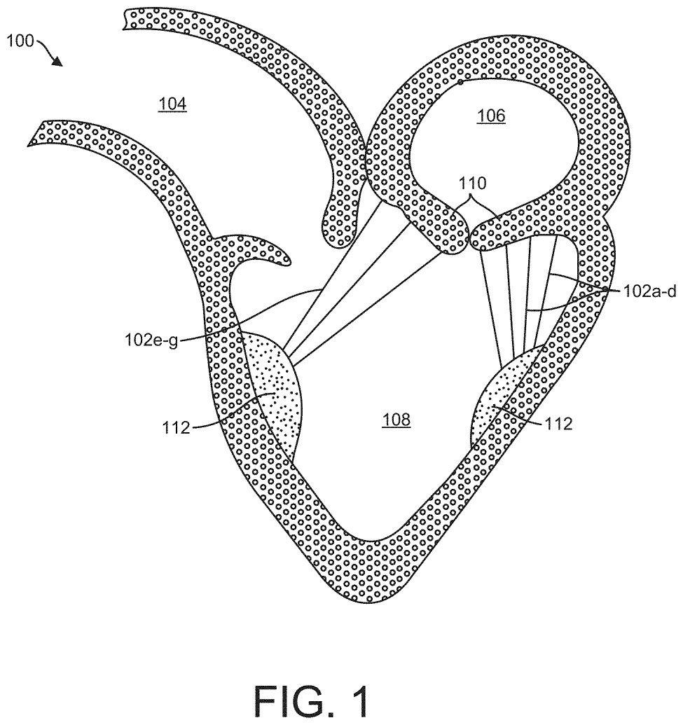

FIG. 1 is an illustration of a patient's heart 100 and chorda tendineae 102a-g in accordance with an embodiment. FIG. 1 shows the left side of the patient's heart 100 that includes the aortic arch 104, left atrium 106, left ventricle 108, with the mitral valve located between the left atrium 106 and the left ventricle 108. The chordae tendineae 102a-g are attached to the leaflets 110 of the mitral valve on one end, and papillary muscles 112 in the left ventricle 108 on the other end.

Stretched, ruptured, or broken chordae tendineae 102a-g may alter functionality of the leaflets 110 of the mitral valve. In these instances, for example, the mitral valve may no longer fully coapt or close. As a result, blood can flow from the left ventricle 108 back into the left atrium 106 (e.g., mitral regurgitation).

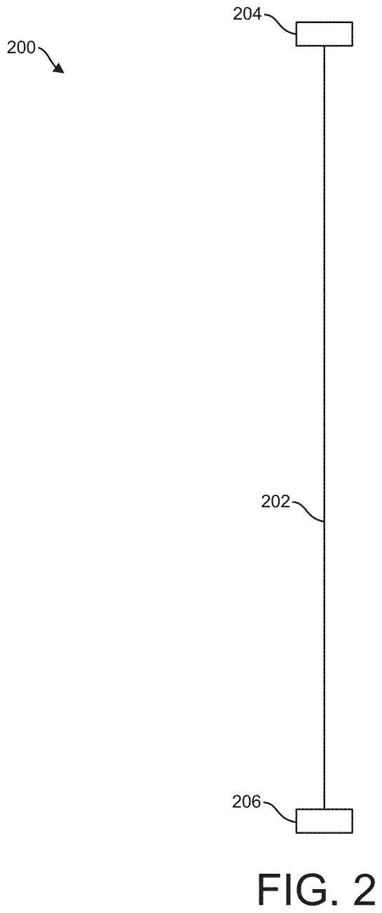

FIG. 2 is an illustration of an example anchoring device 200 in accordance with an embodiment. In certain instances, the anchoring device 200 may include a flexible cord 202, a first attachment member 204 arranged at one end of the flexible cord 202, and a second attachment member 206 arranged at the other end of the flexible cord 202. s described above, the anchoring device 200 may be used for chordae tendineae treatment. In other instances, the anchoring device 200 may be used for tissue anchoring.

According to various examples, the flexible cord 202 is biocompatible and may be made of a material, such as, but not limited to, one or more of polypropylene, Nylon (polyimide), polyester, polyvinylidene fluoride or polyvinylidene difluoride (PVDF), silk, or formed of a fluoropolymer, including without limitation, polytetrafluoroethylene (PTFE) or expanded polytetrafluoroethylene (ePTFE), among other suitable materials. The flexible cord 202 may be formed of suture material, including, but not limited to, monofilament sutures, multifilament and/or braided structures, and woven and non-woven materials. In some examples, the flexible cord 202 is made of a fluoropolymer based suture material, such as that associated with GORE-TEX® Sutures for Chordae Tendineae (“CT”) Treatment. Although synthetic materials are contemplated according to various embodiments, in some examples, the flexible cord 202 may also include natural materials, such as, but not limited to, human or other animal tissues or plant-based material.

The first attachment member 204 and the second attachment member 206 are configured to attach the flexible cord 202 tissue of the heart. The first attachment member 204 and the second attachment member 206 may be anchors that pierce the tissue and retain the flexible cord 202 between a first location and a second location with the first attachment member 204 and the second attachment member 206 piercing and retaining at a surface of or within the tissue at, respectively, the first location and the second location. The first attachment member 204 and the second attachment member 206 may include barbs, fixation helixes, hooks, prongs, or any similar structure.

In certain instances, the flexible cord 202 may be used for treating a defective mitral or tricuspid valve. In these such instances, an apical region of a heart is percutaneously accessed with a catheter-based device. The cardiac valve is repaired by replacing at least one chordae tendineae (e.g., as shown in FIG. 1). The replaced chordae tendineae may include the flexible cord 202, which can also be referred to as a tissue connector due to the flexible cord 202 connecting two portions of the heart tissue. In other instances, the flexible cord 202 may be wrapped about a circumference of the heart or valve annulus may be arranged within a leaflet or tissue. In these instances, the flexible cord 202 slightly compresses one or more structures of the heart to ensure that the leaflets of the valve fully close.

As referenced, the flexible cord 202 can be supplied with one or more anchors or attachment mechanisms. In some examples, the flexible cord 202 is supplied with one or more pledget anchors, for example made of fluoropolymer, such as ePTFE, or any of the materials described in association with the flexible cord 202. Such pledget anchors can be supplied with pre-punched holes or other features and may be used to anchor to one or more valve leaflets or other heart structures. Such anchors may include hook-, corkscrew-, or barbed-designs, among others. In some examples, the flexible cord 202 is supplied with a self-expanding (e.g., such as, but not limited to nitinol (NiTi)) anchor, as described in detail below, configured to assist with anchoring to a heart structure, such as the papillary or ventricular wall. For example, the anchor may be shaped-set NiTi with several leg members that are displaced from a central tube to resist motion.

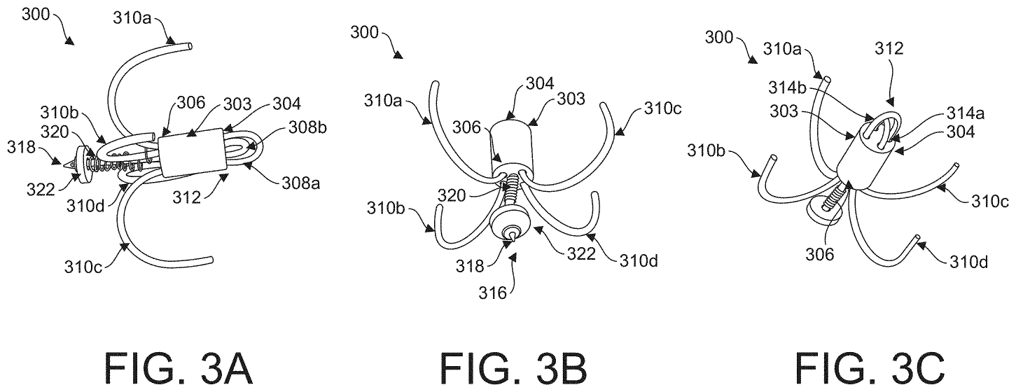

FIG. 3A is a first perspective view of an illustration of an example tissue anchor 300 in accordance with an embodiment. The tissue anchor 300 includes a tubular member 303 having a proximal end 304 and a distal end 306. The tissue anchor 300 also includes at least one least one wire 308a, 308b arranged through the tubular member 303. In certain instances, the one least one wire 308a, 308b includes two wires 308a, 308b. In other instances, the one least one wire 308a, 308b includes a single wire looped through the tubular member 303. In yet other instances, the one least one wire 308a, 308b includes three, four, five, six, or greater number of wires. In certain instances, the tubular member 303 is formed of a biocompatible material such as, but not limited to, polyetheretherketone (PEEK), stainless steel, or a combination thereof.

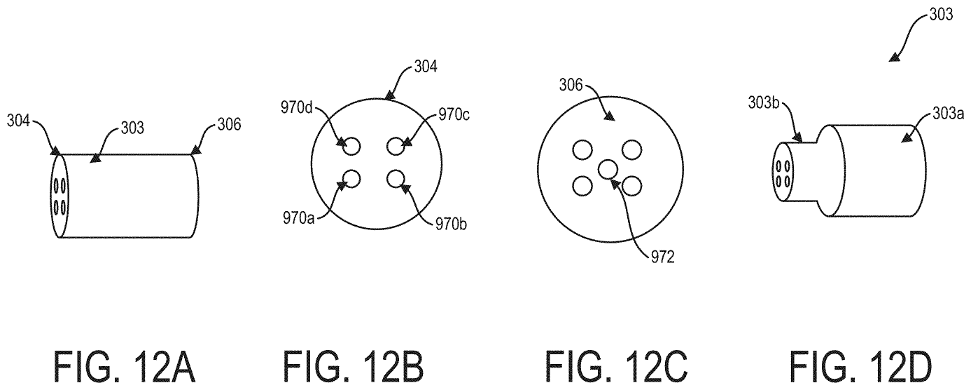

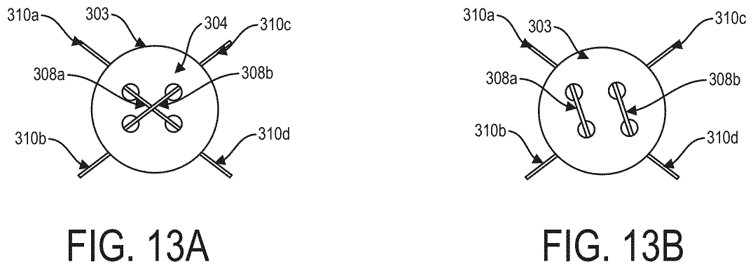

The one least one wire 308a, 308b is arranged to form a plurality of anchors 310a-d that extend from the distal end 306 of the tubular member 306 as shown in further detail in FIG. 3B-C. As shown, the plurality of anchors 310a-d include four anchors. In certain instances, the plurality of anchors 310a-d include, two, three, five, six, or greater of anchors 310a-d. The plurality of anchors 310a-d include a curve that extends the plurality of anchors 310a-d toward the proximal end 304 of the tubular member 303 as shown. In certain instances, the at least one wire 308a, 308b includes two wires 308a, 308b and the plurality of anchors 310a-d includes at least four anchors 310a-d. A wire, as discussed herein, may include mono-filaments, struts, m ulti-filaments, braided strands or other similar structures. In certain instances, the tubular member 303 includes a plurality of lumens (as shown in FIGS. 12A-C and FIG. 13A-B) extending between the proximal end 304 and the distal end 306. In addition, the lumens are configured to maintain an arrangement of the at least one wire 308a, 308b.

The one least one wire 308a, 308b also may form an eyelet 312. The eyelet extends outward from the proximal end 304. In certain instances, a flexible cord, as described in detail above, may be arranged through the eyelet 312 for chordae tendineae treatment. In certain instances, the one least one wire 308a, 308b are arranged to overlap to form the eyelet 312.

As shown in FIGS. 3A and 3C, include curved portions 314a, 314b that extend the one least one wire 308a, 308b outwardly relative to the proximal end 304 of the tubular member 303 and back toward the proximal end 304 of the tubular member 303. In certain instances, the at least one wire 308a, 308b includes two wires 308a, 308b and each of the wire 308a, 308b form one of the curved portions 314a, 314b. In addition, the curved portions 314a, 314b may be arranged approximately perpendicular to one another. Further and as is shown, one of the curved portions 314a, 314b may be arranged above, (e.g., further from the proximal end 304 of the tubular member 303) than the other of the curved portions 314a, 314b.

In certain instances, and as shown in FIG. 3B, the tissue anchor 300 also includes a tissue piercing element 316 extending from the proximal end 304 of the tubular member 303. The tissue piercing element 316 may be arranged between the plurality of anchors 310a-d. In certain instance, the tissue piercing element 316 includes a penetrating tip 318. The penetrating tip 318 may also be configured to anchor within tissue. In addition, the penetrating tip 318 may be helical (e.g., as described in further detail below).

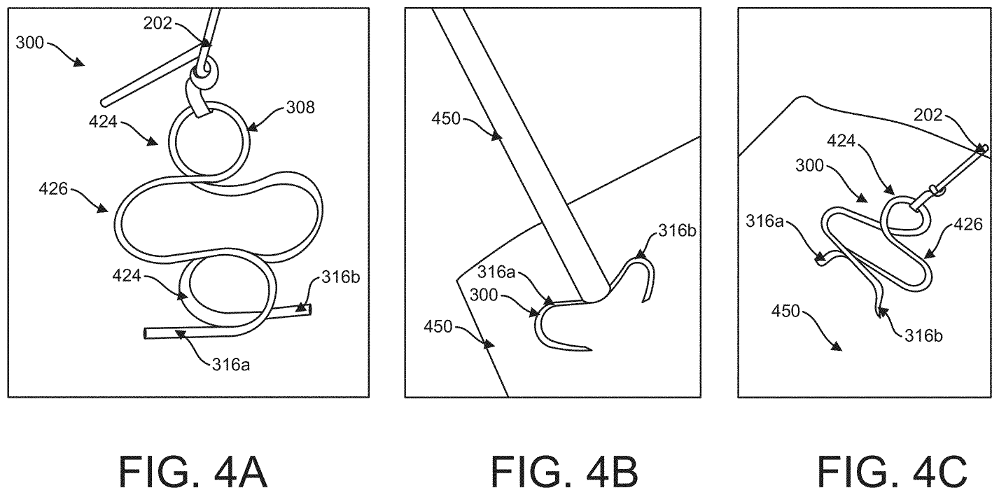

In certain instances, the tissue piercing element 316 includes a longitudinal portion 320 and a depth indicator 322 configured to indicate to a physician embedding of the plurality of anchors 310a-d within tissue. The depth indicator 322 may include a spacer, such as a washer, and a biasing member, such as a spring (e.g. as shown in FIG. 3A), wrapped about the longitudinal portion 320. The spacer may be configured to contact the tissue and force the biasing member toward the distal end 306 of the tubular member 303 to indicate of the plurality of anchors 310a-d within the tissue. In certain instances, the tissue anchor 300 may be arranged within a delivery sheath (e.g., as shown in FIG. 4B) for deployment. The tissue piercing element 316 may be forced into tissue and the biasing member and depth indicator 322 may provide tactile feedback to a physician. Although the tissue anchor 300 shown in FIGS. 3A-C is discussed as being formed of one or more wires 308, the anchors 310a-d may also be formed by a cut-tube pattern or a cut-sheet.

FIG. 4A is a side view of an illustration of another example tissue anchor 300 in accordance with an embodiment. The tissue anchor 300 includes at least one least one wire 308. The at least one least one includes a first circular portion 424, an intermediate portion 426, and one or more tissue piercing elements 316a, 316b.

The first circular portion 424 may be configured to interface with a flexible cord 202, as described in detail above, for chordae tendineae treatment. In addition, the one or more tissue piercing elements 316a, 316b are configured to engage a tissue surface 450 and move between a first configuration, shown in FIG. 4A, and a second configuration, shown in FIG. 4B. In the first configuration, the one or more tissue piercing elements 316a, 316b may be arranged substantially parallel to the tissue surface 450 (e.g., in a non-anchoring configuration). in the second configuration, the one or more tissue piercing elements 316a, 316b may be arranged non-parallel to the tissue surface 450.

The intermediate portion 426 is configured to move the one or more tissue piercing elements 316a, 316b from the first configuration to the second configuration in response to a reduction in diameter of the intermediate portion 426. In certain instances, the intermediate portion 426 is substantially oval in shape as shown in FIG. 4A. The intermediate portion 426 may be configured to reduce in diameter through arrangement within a delivery sheath 452 as shown in FIG. 4B. The delivery sheath 452 is configured to house the tissue anchor 300. The delivery sheath 452 is configured to reduce the intermediate portion 426 in diameter and unconstrain the middle portion to allow movement of the one or more tissue piercing elements 316a, 316b from the first configuration to the second configuration

The reduction in diameter of the intermediate portion 426 moves the one or more tissue piercing elements 316a, 316b from the non-engagement configuration (e.g., substantially parallel to a long axis of the intermediate portion 426) to be arranged non-parallel to the tissue surface 450 (e.g., substantially perpendicular to the long axis of the intermediate portion 426) as shown in FIG. 4B. The one or more tissue piercing elements 316a, 316b are configured to grip the tissue surface 450 in response to movement from the first configuration to the second configuration. The one or more tissue piercing elements 316a, 316b may pierce the tissue surface 450 to secure the tissue anchor 300 in place. As shown in FIG. 4C, the one or more tissue piercing elements 316a, 316b remain in the tissue surface 450 after removal of the tissue surface 450. When engaged, the intermediate portion 426 moves back toward the substantially oval or oblong shape. The tissue piercing elements 316a, 316b remain non-parallel to the tissue surface 450.

In addition, the at least one wire 308 includes a second circular portion 456, as shown in FIG. 4A, and the tissue piercing elements 316a, 316b extend from the second circular portion 456. In the second engagement configuration, the tissue piercing elements 316a, 316b are spread apart and the second circular portion 456 expands outwardly as shown in FIG. 4C.

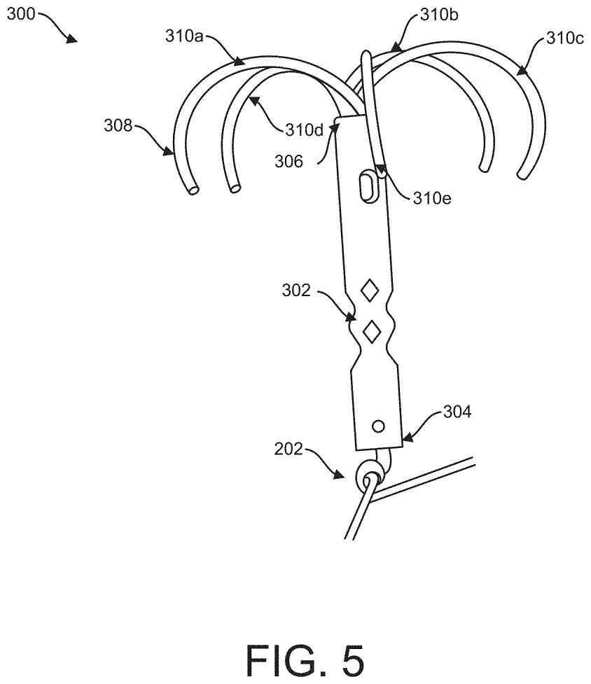

FIG. 5 is a first perspective view of an illustration of an example tissue anchor 300 in accordance with an embodiment. The tissue anchor 300 includes a tubular member 303 having a proximal end 304 and a distal end 306. The tissue anchor 300 also includes one or more one wires 308 arranged through the tubular member 303. The tissue anchor 300 also includes plurality of anchors 310a-e. In certain instances, the number of wires 308 is equal to the anchors 310a-e. In other number of wires 308 is half the number of anchors 310a-e with the wires being arranged through the tubular member 303 and folded back to provide one of the anchors 310a-e at one end, and another of the anchors 310a-e at another end of each of the wires 308. The anchors 310a-e may be configured and arranged as described in further detail with reference to FIGS. 3A-B. In addition, the tissue anchor may also include a tissue piercing element as described above.

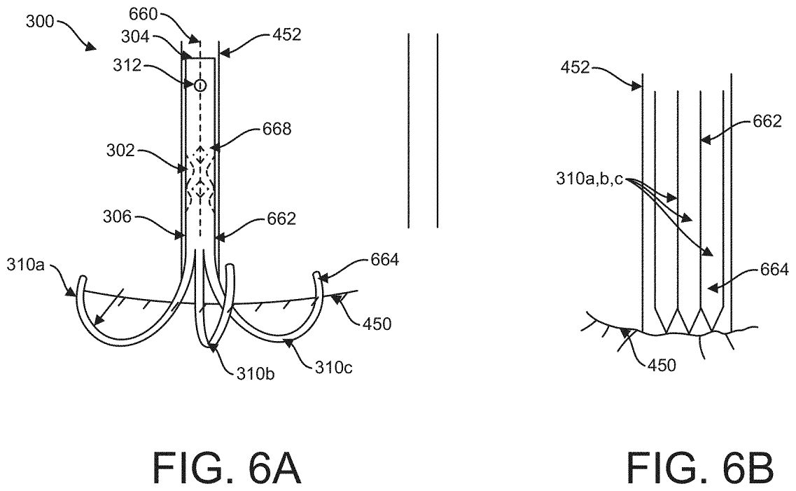

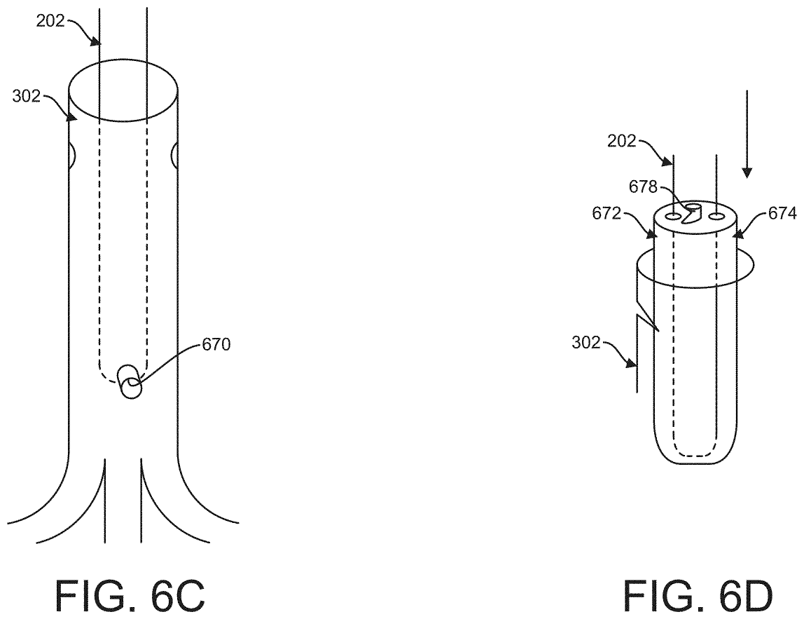

FIG. 6A is an illustration of an example tissue anchor 300 in a deployed configuration, in accordance with an embodiment. The tissue anchor 300 may include a cylindrical portion 302 having a longitudinal axis 660, a proximal end 304 and a distal end 306. The tissue anchor 300 also includes a plurality of anchors 310a-c configured to secure the cylindrical portion 302 under a tissue surface 350.

Each of the plurality of anchors 310a-c includes a substantially linear section 662 extending parallel from the distal end 306 of the cylindrical portion 302 to the longitudinal axis 660. In addition, the plurality of anchors 310a-c includes a curved section 664 extending from the substantially linear section 662. The curved section 664 of each of the plurality of anchors 310a-c may be configured to align with the substantially linear section 662 relative to the longitudinal axis 660 in a delivery configuration, shown in FIG. 6B and curve radially outwardly relative to the longitudinal axis 660 and toward the distal end 306 of the cylindrical portion 302 in a deployed configuration as shown in FIG. 6A. As shown in FIG. 6A, the cylindrical portion 302 may be arranged within the delivery sheath 452 during delivery.

In certain instances, the cylindrical portion 302 includes one or more notches 668 in an exterior surface of the cylindrical portion. The notches 668 are cut-outs or remove portions of the material of the cylindrical portion 302. The notches 668 may facilitate flexibility of the cylindrical portion 302. In certain instances, the plurality of anchors 310a-c and the cylindrical portion 302 are formed a unitary structure such as a cut-tube. In addition, the cylindrical portion 302 may include an eyelet 312 that may be configured to interface with a flexible cord 202, as described in detail above, for chordae tendineae treatment.

In certain instances, the cylindrical portion 302 also includes a flexible cord pivot 670. The flexible cord 202, as shown in FIG. 6C, may be arranged within the cylindrical portion 302 and extend into about the flexible cord pivot 670. The flexible cord pivot 670 may facilitate securing the flexible cord 202 to the cylindrical portion 302.

In certain instances, the tissue anchor 300 may also include an adjustable mechanism 672. As shown in FIG. 6D, the adjustable mechanism 672 may be arranged within the cylindrical portion 302. The adjustable mechanism 672 may include an insert 674 that the flexible cord 202 may be arranged through as shown. The insert 674 may be secured within the cylindrical portion 302 by an adhesive or interference fit. The adjustment mechanism 672 may be configured to adjust a length of the flexible cord 202. Tension may be applied to the flexible cord 202 to adjust a length of the flexible cord 202. The adjustment mechanism 672 may include a blade 678 to trim the length of the flexible cord 202. The flexible cord 202 may be arranged and secured within the insert 674.

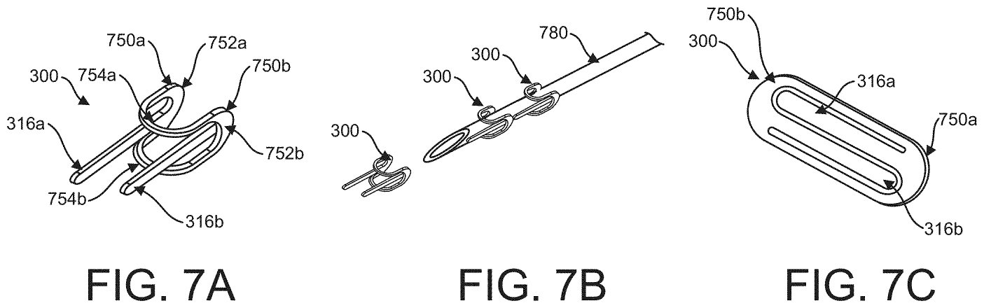

FIG. 7A is a perspective view of an illustration of another example tissue anchor 300 in accordance with an embodiment. The tissue anchor 300, which may be formed from a wire or a cut-tube. The tissue anchor 300 may include a first anchor section 750a including a first curved section 752a and a first tissue piercing element 316a. extending longitudinally from an interior surface of an apex of the first curved section 752a. The tissue anchor 300 may also include a second anchor section 750b including a second curved section 752b and a second tissue piercing element 316b extending longitudinally from an interior surface of an apex of the second curved section 752b. The apices may be the curved interior surfaces of the curved sections 752a, 752b.

The tissue anchor 300 may also include a first transition section 754a arranged between and extending perpendicular to the first anchor section 750a and the second anchor section 750b in a first plane. In addition, the tissue anchor 300 may also include a second transition section 754b arranged between and extending perpendicular to the first anchor section 750a and the second anchor section 750b in a second plane. In certain instances, the first plane and the second plane are spaced apart from one another.

In certain instances, the first transition section 754a and the second transition section 754b are parallel to one another. In addition, the first anchor section 750a and the second anchor section 750b may be arranged parallel to one another. Further, at least one of the first transition section 754a and the second transition includes 754b a curvature as is shown in FIG. 7A.

FIG. 7B is a perspective view of the tissue anchor 300, shown in FIG. 7A, arranged with a needle 780 in accordance with an embodiment. The needle 780 may be used to implant the tissue anchor 300. The needle 780 may be arranged through and between the first anchor section 750a, the second anchor section 750b, the first transition section 754a, and the second transition section 754b. In addition, one or more of the first anchor section 750a, the second anchor section 750b, the first transition section 754a, and the second transition section 754b may be configured to interface with a flexible cord 202, as described in detail above, for chordae tendineae treatment.

FIG. 7C is a deployed view of the tissue anchor 300, shown in FIGS. 7A-B, in accordance with an embodiment. In certain instances, the first anchor section 750a, the second anchor section 750b, the first transition section 754a, and the second transition section 754b may be configured to deploy to within a substantially common plane as is shown. The arrangement shown in FIG. 7C may also be a cut-pattern for the tissue anchor 300 shown.

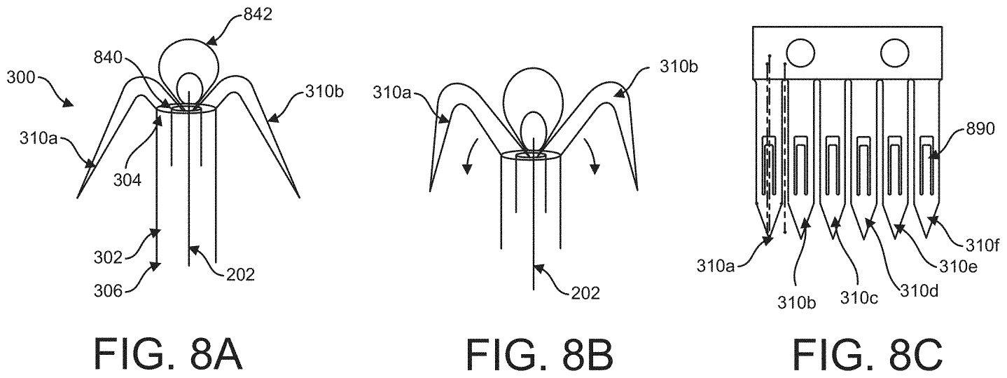

FIG. 8A is an illustration of a tissue anchor 300 in a first configuration, in a delivery configuration in accordance with an embodiment. The tissue anchor 300 shown includes a cylindrical portion 302 having a lumen 840, a proximal end 304 and a distal end 306.

The tissue anchor 300 includes a plurality of anchors 310a, 310b (although the tissue anchor 300 may include more anchors as shown in FIG. 8C) extending from the lumen 840 and radially outwardly relative to an exterior surface of the cylindrical portion 302. The tissue anchor also includes a ball 842 arranged at or adjacent to the distal end 306 of the cylindrical portion 302. A flexible cord 202 may extend within the lumen 840 of the cylindrical portion 302 and may be coupled to the ball 842. The flexible cord 202, in certain instances, is configured to withdraw the ball 842 toward the proximal end 304 and at least partially within the lumen 840 to alter a configuration of the plurality of anchors 310a, 310 as is shown in FIG. 8B.

In response to tension, the plurality of anchors 310a, 310b may splay open more. The plurality of anchors 310a, 310b may be configured to secure within tissue. In addition, the flexible cord 202 may be for chordae tendineae treatment. The ball 842 contacting the anchors 310a, 310b may cause the movement of the anchors 310a, 310b.

FIG. 8C is an illustration of a cut pattern for a portion of the tissue anchor 300, shown in FIGS. 8A-B, in accordance with an embodiment. As shown in the cut pattern, the tissue anchor 300 may include a greater number of anchors 310a-f than shown in FIGS. 8A-B. The tissue anchor 300 may deploy upward or downward relative to a tissue wall (as can the other tissue anchors 300 discussed herein). In addition, the anchors 310a-f may include greater or less widths. In addition, the anchors 310a-f may include sub-anchors 890 (one highlighted for ease of illustration). The sub-anchors 890 may deploy as a barb in a direction opposite that of the anchors 310a-f. This may be beneficial for deployment when the anchors 310a-f are arranged upward (with the tissue anchor 300 being embedded or within) the tissue surface.

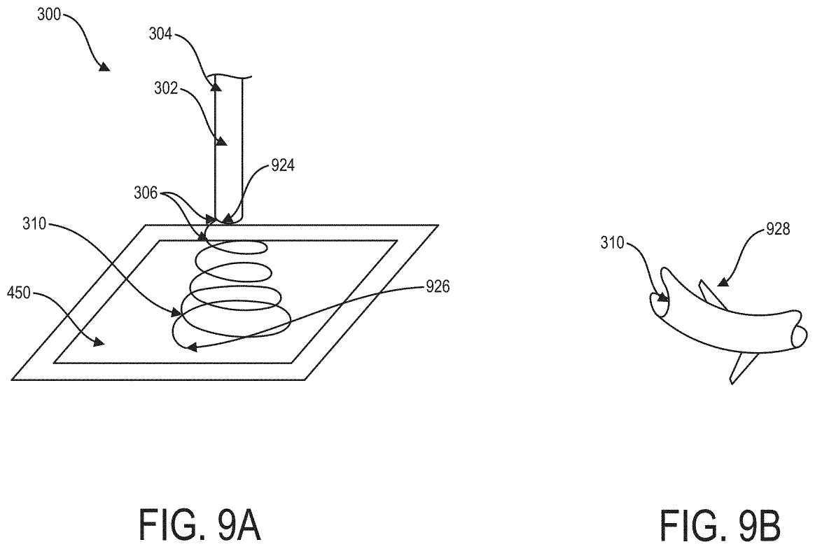

FIG. 9A is a perspective view of an illustration of another example tissue anchor 300 in accordance with an embodiment. The tissue anchor 300 may include a cylindrical portion 302 having a proximal end 304 and a distal end 306. The tissue anchor 300 may also include an anchor element 310 configured to secure the cylindrical portion 302 to or under a tissue surface 450.

As shown, the anchor element 310 includes a helical structure having a proximal end 924 arranged at the distal end 306 of the cylindrical portion 302 and including a plurality of loops increasing in diameter from the proximal end of the helical structure to a distal end 926 of the helical structure. The helical structure increasing in diameter may increase surface area engagement of the anchor element 310. In addition, the anchor element 310 (or any anchor element discussed herein) may include one or more barbs 928 arranged on an exterior surface of the helical structure. The barbs 928 may further grip the tissue surface 450 to secure the anchor element 310 therein. The cylindrical portion 302 may be configured to interface with a flexible cord 202, as described in detail above, for chordae tendineae treatment.

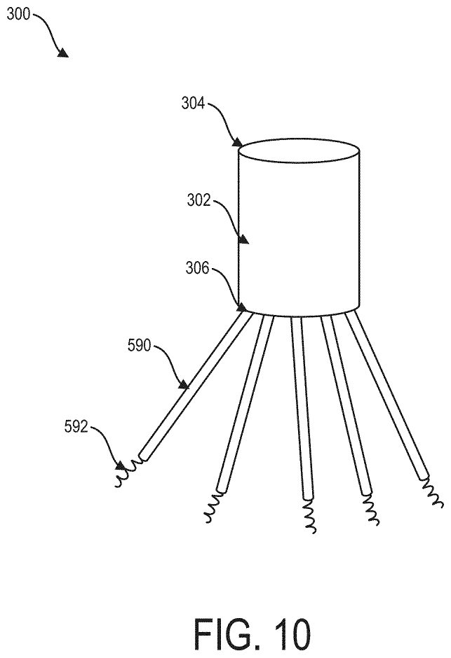

FIG. 10 is a perspective view of an illustration of another example tissue anchor 300 in accordance with an embodiment. The tissue anchor 300 may include a cylindrical portion 302 having a proximal end 304 and a distal end 306. In addition, a plurality of substantially linear sections 590 (one highlighted for ease of illustration) extending from the distal end 306 of the cylindrical portion 302.

In addition, the tissue anchor 300 shown in FIG. 10 may include a plurality of helical anchors 592 arranged at distal ends of each of the plurality of substantially linear sections 590. The helical anchors 592 may be configured to secure the cylindrical portion 302 to a tissue wall. Any of the tissue anchors 300 discussed herein may include the additional helical anchors 592 arranged at a distal end of the anchors (e.g., in place of penetrating tip 318).

In certain instances, the cylindrical portion 302 may be configured to interface with a flexible cord 202, as described in detail above, configured for chordae tendineae treatment.

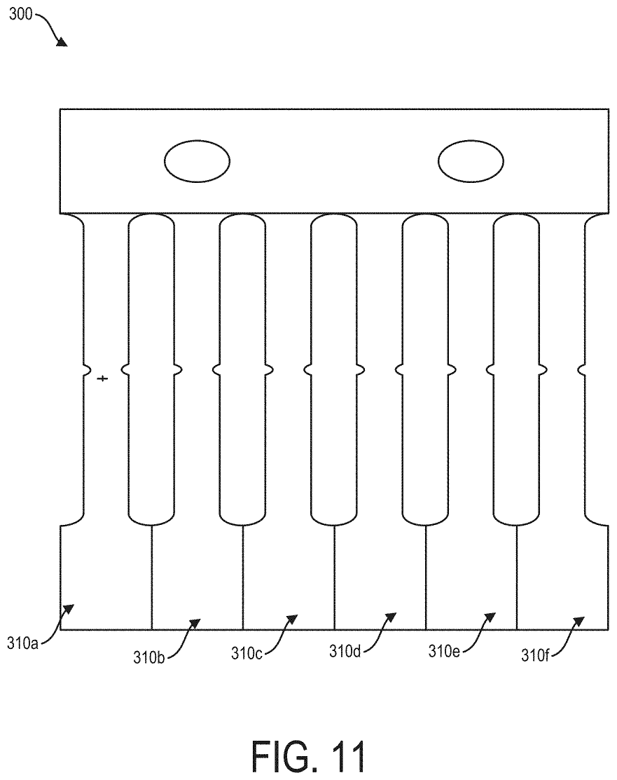

FIG. 11 is a view of an example cut-pattern for a tissue anchor 300 in accordance with an embodiment. The tissue anchor 300 may include a plurality of anchors 310a-f as shown. The anchors 310a-f may include blunt or non-pointed ends.

FIG. 12A is a side view of an example tubular member 303 for a tissue anchor in accordance with an embodiment. As described in detail above with reference to FIGS. 3A-C, the tubular member 303 includes a proximal end 304 and a distal end 306. The tissue anchor 300 also includes at least one least one wire (or struts formed from a cut-tube or cut-sheet) arranged through the tubular member 303.

As shown in FIGS. 12B and 12C, the tubular member 303 includes a plurality of lumens 970a-d extending between the proximal end 304 and the distal end 306. In addition, the lumens 970a-d are configured to maintain an arrangement of the at least one wire 308a, 308b. As also shown in viewing the distal end 306 of the tubular member 303, the tubular member 303 may include an additional opening 972 for the tissue piercing element 316. In certain instances and as is shown, the addition opening 972 may only be arranged within the distal end 306 of the tubular member 303 and not extend to the proximal end 304.

FIG. 12D is a side view of another example configuration of the tubular member 303, shown in FIGS. 12A-C, in accordance with an embodiment. in certain instances, the tubular member 303 may include two different diameter sections. A first section 303a of the tubular member 303 may be larger than a second section 303b of the tubular member 303. The second section 303b may be the proximal end 304 of the tubular member 303 or the distal end 306 of the tubular member 303. In certain instances, the second section 303b is configured to engage a delivery system.

FIG. 13A is a proximal end view of an example tubular member 303 and at least one wire 308a, 308b arranged through the tubular member, in accordance with an embodiment. As described in detail above with reference to FIGS. 3A-C, the tubular member 303 includes a proximal end 304 and a distal end 306. The tissue anchor 300 also includes at least one least one wire 308a, 308b arranged through the tubular member 303. In certain instances, the one least one wire 308a, 308b includes two wires 308a, 308b. In other instances, the one least one wire 308a, 308b includes a single wire looped through the tubular member 303 or any number of wires arranged through the tubular member 303 as discussed in detail above.

As shown in FIG. 13A, the wires 308a, 308b cross one another at a proximal end 304 of the tubular member 303. The wires 308a, 308b are arranged through the tubular member 303 to form a plurality of anchors 310a-d. In other instances and as shown in FIG. 13B, the wires 308a, 308b are non-overlapping at the proximal end 304 of the tubular member 303. The wires 308a, 308b are arranged through the tubular member 303 to form the plurality of anchors 310a-d.

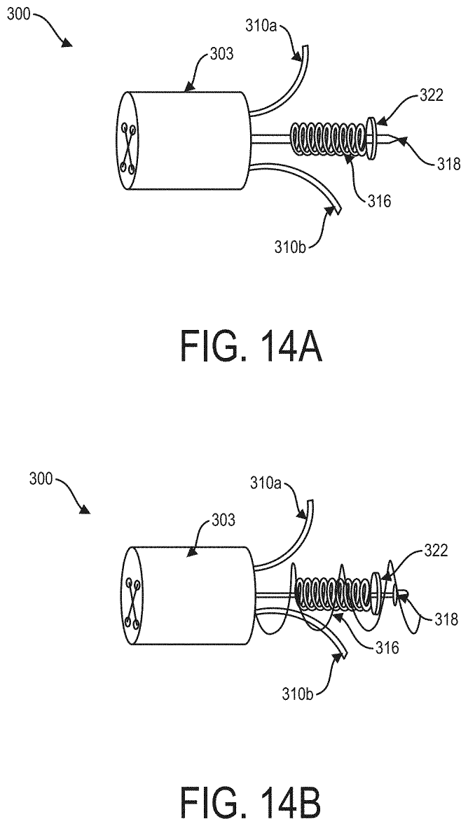

FIG. 14A is a side view of an example tissue anchor 300 and depth indicator 322, in accordance with an embodiment. The tissue anchor 300 includes a tubular member 303 and at least one least one wire 308a, 308b arranged through the tubular member 303. In certain instances, the one least one wire 308a, 308b includes two wires 308a, 308b. In other instances, the one least one wire 308a, 308b includes a single wire looped through the tubular member 303. In yet other instances, the one least one wire 308a, 308b includes three, four, five, six, or greater number of wires.

The one least one wire 308a, 308b is arranged to form a plurality of anchors 310a-b that extend from the distal end 306 of the tubular member 303.

The tissue anchor 300 also includes a tissue piercing element 316 extending from the tubular member 303. The tissue piercing element 316 may be arranged between the plurality of anchors 310a-b. In certain instance, the tissue piercing element 316 includes a penetrating tip 318. The penetrating tip 318 may also be configured to anchor within tissue. In addition, the penetrating tip 318 may be helical (e.g., as described in further detail below).

In certain instances, the depth indicator 322 is configured to indicate to a physician embedding of the plurality of anchors 310a-d within tissue. The tissue piercing element 316 may be forced into tissue and the depth indicator 322 may provide tactile feedback to a physician. The tissue piercing element 316 be formed of a wire or a portion of a cut-tube or cut-sheet in certain instances. in addition, the tissue piercing element 316 may be substantially linear as shown in FIG. 14A, or in other instances, the tissue piercing element 316 may be helical as shown in FIG. 14B.

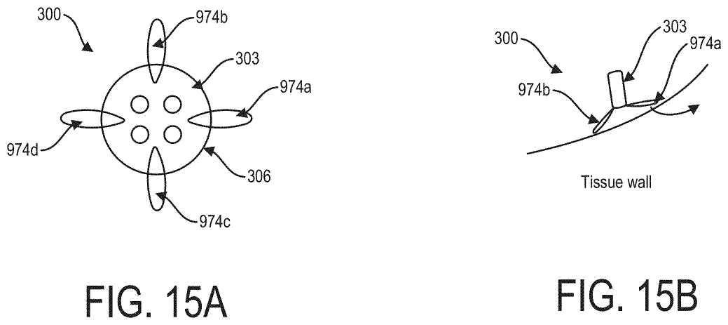

FIG. 15A is a distal end view of an example tubular member 303 and a location feedback mechanism 974a-d, in accordance with an embodiment. In certain instances, the tubular member 303 may include the location feedback mechanism 974a-d arranged at a distal end 306 of the tubular member 303. The location feedback mechanism 974a may be one or more wire or strut elements extending radially outwardly from the distal end 306. In certain instances, the location feedback mechanism 974a-d may take the form of the anchors 310a-d, discussed in detail above, or the location feedback mechanism 974a-d may be structures additional to the anchors.

FIG. 15B is a view of the tubular member 303 and the location feedback mechanism, shown in FIG. 15A, engaging a tissue wall, in accordance with an embodiment. The location feedback mechanism includes feedback elements 974a-d (e.g., wires, struts, cut-tube elements, cut-sheet elements) or are configured to indicate visual feedback of the tissue wall angle. As shown, the angle of the location feedback elements 974a-d relative to the tubular member 303 indicates the angle or slope of the tissue wall.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A tissue anchor comprising:
a tubular member having a proximal end and a distal end;
at least one wire arranged through the tubular member to form:
a plurality of anchors extending from the distal end of the tubular member, each anchor defining a curve extending in a direction toward the proximal end, and
an eyelet extending from the proximal end; and
a tissue piercing element extending from the proximal end of the tubular member and arranged adjacent to the plurality of anchors.

2. The tissue anchor of claim 1, wherein the at least one wire includes at least two wires arranged through the tubular member, and the at least two wires overlap to form the eyelet.

3. The tissue anchor of claim 2, wherein the at least two wires form the plurality of anchors and the plurality of anchors includes at least four anchors.

4. The tissue anchor of claim 1, wherein the tubular member includes a plurality of lumens extending between the proximal end and the distal end, and the lumens are configured to maintain an arrangement of the at least one wire.

5. The tissue anchor of claim 1, wherein the tissue piercing element includes a longitudinal portion and a depth indicator configured to provide tactile feedback to a physician embedding of the plurality of anchors within tissue.

6. The tissue anchor of claim 5, wherein the depth indicator includes a spacer and a biasing member arranged about the longitudinal portion of the tissue piercing element, the spacer is configured to contact tissue and urge the biasing member toward the distal end of the tubular member, the degree of which is configured to indicate the extent to which the plurality of anchors extend within the tissue.

7. The tissue anchor of claim 1, wherein the tubular member comprises at least one of PEEK and stainless steel.

8. The tissue anchor of claim 1, further comprising a flexible cord arranged through the eyelet for coupling the tissue anchor to tissue.

9. The tissue anchor of claim 1, further comprising a location feedback mechanism arranged at the distal end of the tubular member and configured to indicate a slope or angle of a tissue wall.

10. A tissue anchor comprising:
at least one wire arranged to form:
a first circular portion configured to interface with a flexible cord,
one or more tissue piercing elements configured to engage a tissue surface and move between a first configuration in which the one or more tissue piercing elements are arranged substantially parallel to the tissue surface and a second configuration where the one or more tissue piercing elements are arranged non-parallel to the tissue surface, and
an intermediate portion configured to move the one or more tissue piercing elements from the first configuration to the second configuration in response to a reduction in diameter of the middle portion.

11. The tissue anchor of claim 10, wherein the intermediate portion is substantially oval in shape.

12. The tissue anchor of claim 10, wherein the at least one wire includes a second circular portion and the one or more tissue piercing elements extend from the second circular portion.

13. The tissue anchor of claim 10, further comprising a delivery sheath configured to reduce the intermediate portion in diameter and unconstrain the intermediate portion allow movement of the one or more tissue piercing elements from the first configuration to the second configuration.

14. The tissue anchor of claim 13, wherein the one or more tissue piercing elements are configured to grip the tissue surface in response to movement from the first configuration to the second configuration.

15. The tissue anchor of claim 10, wherein the flexible cord is configured for coupling the tissue anchor to tissue.

16. A tissue anchor comprising:
a cylindrical portion having a longitudinal axis, a proximal end and a distal end;
and
a plurality of anchors configured to secure the cylindrical portion under a tissue surface, each of the plurality of anchors including:
a substantially linear section extending from the distal end of the cylindrical portion parallel to the longitudinal axis, and
a curved section extending from the substantially linear section and configured to align with the substantially linear section relative to the longitudinal axis in a delivery configuration and curve radially outwardly relative to the longitudinal axis and toward the distal end of the cylindrical portion in a deployed configuration.

17. The tissue anchor of claim 16, wherein the cylindrical portion includes one or more notches in an exterior surface of the cylindrical portion.

18. The tissue anchor of claim 17, wherein the one or more notches are configured to facilitate flexibility of the cylindrical portion.

19. The tissue anchor of claim 16, further comprising a flexible cord arranged through an opening in the cylindrical portion configured for coupling the tissue anchor to tissue.

20. The tissue anchor of claim 19, wherein the cylindrical portion includes an adjustment mechanism configured to adjust a length of the flexible cord.

21. A tissue anchor comprising:
a first anchor section including a first curved section and a first tissue piercing element extending longitudinally from an interior surface of an apex the first curved section;
a second anchor section including a second curved section and a second tissue piercing element extending longitudinally from an interior surface of an apex of the second curved section;
a first transition section arranged between and extending perpendicular to the first anchor section and the second anchor section in a first plane; and
a second transition section arranged between and extending perpendicular to the first anchor section and the second anchor section in a second plane.

22. The tissue anchor of claim 21, wherein the first plane and the second plane are spaced apart from one another.

23. The tissue anchor of claim 21, wherein at least one of the first transition section and the second transition defines a curve.

24. The tissue anchor of claim 21, wherein the first anchor section, the second anchor section, the first transition section, and the second transition are configured to deploy to within a substantially common plane.

25. The tissue anchor of claim 21, further comprising a flexible cord coupled to at least one of the first anchor section, the second anchor section, the first transition section, and the second transition for coupling the tissue anchor to tissue.

26. A tissue anchor comprising:
a cylindrical portion having a proximal end and a distal end and a lumen therethrough;
a plurality of anchors extending from the lumen and radially outwardly relative to an exterior surface of the cylindrical portion;
a ball arranged at or adjacent to the distal end of the cylindrical portion; and
a flexible cord extending within the lumen of the cylindrical portion and coupled to the ball, the flexible cord being configured to withdraw the ball toward the proximal end and engage the plurality of anchors to alter a configuration of the plurality of anchors.

27. The tissue anchor of claim 26, wherein the ball is configured to at least partially withdraw into the lumen to alter the configuration of the plurality of anchors.

28. A tissue anchor comprising:
a cylindrical portion having a proximal end and a distal end; and
an anchor element configured to secure the cylindrical portion to or under a tissue surface, the anchor element having a helical structure having a proximal end arranged at the distal end of the cylindrical portion and including a plurality of loops of increasing diameter from the proximal end of the helical structure to a distal end of the helical structure.

29. The tissue anchor of claim 28, further comprising one or more barbs arranged on an exterior surface of the helical structure.

30. The tissue anchor of claim 28, further comprising a flexible cord coupled to the cylindrical portion configured for coupling the tissue anchor to tissue.

31. A tissue anchor comprising:
a cylindrical portion having a proximal end and a distal end;
a plurality of substantially linear sections extending from the distal end of the cylindrical portion; and
a plurality of helical anchors arranged at distal ends of each of the plurality of substantially linear sections configured to secure the cylindrical portion to a tissue wall.

32. The tissue anchor of claim 31, further comprising a flexible cord coupled to the cylindrical portion configured for coupling the tissue anchor to tissue.

33. A method for chordae tendineae repair includes: arranging a flexible cord through a leaflet and anchoring a first end of the flexible cord to the leaflet; coupling a second end of the flexible cord to the tissue anchor; and anchoring the tissue anchor of claim 1 to tissue of a heart.
